(19) United States
(12) Patent Application Publication
(10) Pub. No.: US 2024/0164788 A1
(43) Pub. Date: May 23, 2024

(54) THERAPEUTIC TREATMENT DEVICE WITH BRAIDED-STRAND MICROVALVE OCCLUDER HAVING MODIFIED FILTER COATING

(71) Applicant: TriSalus Life Sciences, Inc., Westminster, CO (US)

(72) Inventors: Erik Dean Olson, Castle Rock, CO (US); Michael Brick Markham, Boulder, CO (US); David Benjamin Jaroch, Arvada, CO (US)

(73) Assignee: TriSalus Life Sciences, Inc., Westminster, CO (US)

(21) Appl. No.: 17/992,433

(22) Filed: Nov. 22, 2022

(51) Int. Cl. A61B 17/12

(52) U.S. Cl. CPC A61B 17/12163; A61B 17/12109; A61B 17/12172

(57) ABSTRACT

A therapeutic device includes a catheter and a temporary vessel occluder. The vessel occluder has a plurality of elastic strands extending in a tubular arrangement from the first end to the second, a primer on the strands, and a polymer filter coating over the primer. The primer is unevenly distributed to the plurality of elastic strands between the plurality of elastic strands. The filter coated is applied over the primer and on the strands. A portion of the polymer coating may be removed.

### CROSS-REFERENCE TO RELATED PATENTS

This application is related to co-owned U.S. Pat. Nos. 8,696,698 and 10,588,636, which are hereby incorporated by reference herein in their entireties.

### BACKGROUND OF THE INVENTION

### 1. Field of Invention

The disclosure relates generally to infusion catheters for delivering a therapeutic treatment into a blood vessel. More particularly, this disclosure relates to infusion catheters having microvalves at the distal end thereof to increase penetration of the therapeutic treatment into targeted blood vessels and reduce reflux of the therapeutic treatment into non-targeted blood vessels.

### 2. State of the Art

Intravascular therapeutic treatments are often clinically delivered to treat a wide range of diseases. By way of example, intravascular embolization, chemo-embolization, and radio-embolization therapies are used to treat a range of diseases, such as hypervascular liver tumors, uterine fibroids, secondary cancer metastasis in the liver, pre-operative treatment of hypervascular menangiomas in the brain and bronchial artery embolization for hemoptysis.

Non-targeted delivery of various therapies can lead to adverse events and morbidity. In addition, non-targeted delivery suggests that the intended target of the delivery is not receiving the full dose of therapy.

Infusion with a standard infusion microcatheter allows bi-directional flow. That is, the use of a microcatheter to infuse a therapeutic agent allows blood and the infused therapeutic agent to move forward in addition to allowing blood and the therapeutic agent to be pushed backward (reflux). Reflux of a therapeutic agent causes non-target damage to surrounding healthy organs. In interventional oncology therapeutic procedures, the goal is to bombard a cancer tumor with either radiation or chemotherapy. It is important to maintain forward flow throughout the entire vascular tree in the target organ in order to deliver therapies into the distal vasculature, where the therapy can be most effective. This issue is amplified in hypovascular tumors or in patients who have undergone chemotherapy, where slow flow limits the dose of therapeutic agent delivered and reflux of agents to non-target tissue can happen well before the physician has delivered the desired dose.

The pressure in a vessel at multiple locations in the vascular tree changes during a therapeutic infusion procedure. Initially, the pressure is high proximally, and decreases over the length of the vessel. Forward flow of therapy occurs when there is a pressure drop. If there is no pressure drop over a length of vessel, therapy does not flow downstream. If there is a higher pressure at one location, such as at the orifice of a catheter, the therapeutic therapy flows in a direction toward lower pressure. If the pressure generated at the orifice of an infusion catheter is larger than the pressure in the vessel proximal to the catheter orifice, some portion of the infused therapeutic therapy travels up stream (reflux) into non-target vessels and non-target organs. This phenomenon can happen even in vessels with strong forward flow if the infusion pressure (pressure at the orifice of the catheter) is sufficiently high.

In clinical practice with a standard infusion catheter, the physician attempts to infuse the therapeutic agent with pressure that does not cause reflux. In doing this, the physician slows the infusion rate (and infusion pressure) or stops the infusion completely. The clinical impact of current infusion catheters and techniques is two fold: low doses of the therapeutic agent is delivered and there is poor distal penetration into the target vessels.

Additionally, reflux can be a time-sensitive phenomenon. Sometimes, reflux occurs as a response to an injection of the therapeutic agent, where the reflux occurs rapidly (e.g., in the time-scale of milliseconds) in a manner which is too fast for a human operator to respond. Also, reflux can happen momentarily, followed by a temporary resumption of forward flow in the blood vessel, only to be followed by additional reflux.

Various devices have been proposed to increase distal penetration while preventing reflux. For example, co-owned U.S. Pat. No. 8,696,698, which has been incorporated by reference herein, describes a microvalve infusion system for infusing a therapeutic agent that has a dynamically adjustably filter valve coupled at a distal end of a delivery catheter. The delivery catheter and filter valve self-expand when deployed from a delivery catheter. The filter valve is naturally spring biased by its construction of filamentary elements to automatically partially expand within a vessel when it is deployed from the outer catheter, and is coated with a porous polymer coating that has a pore size sufficiently small to filter or block a therapeutic agent. In view of the construction, upon infusion, an increase in fluid pressure results within the filter valve and causes the filter valve to open, extend across a vessel, and thereby prevent reflux of the infused therapeutic agent. In addition, as the fluid is pressurized through the delivery catheter and into the filter valve, the downstream pressure in the vessel is increased which facilitates maximum uptake into the target tissue for therapeutically delivered agents. Further, the filter valve is responsive to local pressure about the valve which thereby enables substantially unrestricted forward flow of blood in the vessel, and reduces or stops reflux (regurgitation or backward flow) of therapeutic agents which are introduced into the blood.

However, devices in U.S. Pat. No. 8,696,698 have certain design characteristics that may not always be advantageous for a given situation. The filter valve devices disclosed are generally well-adapted where tracking the occluder into small vessels in not a significant requirement; trackability in tortuous branching vasculature can be limited. The distal end of the device in a collapsed, undeployed state is defined by the size of the deployment catheter through which the occluder must be advanced into a vessel, which can be significantly larger than the catheter that supports the filter valve and significantly larger than the outer diameter of a guidewire used to the guide the microvalve to the target location within the vessel. As such, tracking the filter valve into the smaller vascular branches may not be optimal. In addition, once the device is tracked to a treatment location, deployment of the filter valve requires that the frictional force between the filter valve and the outer deployment catheter be overcome.

Co-owned U.S. Pat. No. 10,588,636, previously incorporated herein, describes a microvalve infusion system for infusing a therapeutic agent and which addresses device trackability. Referring to Prior Art FIG. 1, the system 10 includes a flexible infusion catheter 12 having a hub 14 at a proximal end 16 and a filter valve occluder 18 coupled to a distal end 20. The filter valve occluder 18 includes braided elastic strands 22 that each include a proximal portion 24, a central portion 26, and a distal portion 28. The proximal portions 24 are attached circumferentially about at an outer surface 30 of the catheter 12 at a location proximal of a distal orifice 32 of the catheter, the central portions 26 extend radially outward and toward the orifice 32, and the distal portions 28 of the strands are inverted back into the filter valve occluder 18 and coupled circumferentially about the outer surface 30 of the catheter 12. The proximal and central portions 24, 26 of the strands 22 are coated in a polymeric filter coating 34 that extends between and across the strands 18. The distal portions 28 of the strands 22 are uncoated in the polymeric filter coating.

### SUMMARY OF THE INVENTION

An infusion device is provided that includes a catheter and a microvalve. The catheter has a proximal end, a distal end with a distal tip, and a lumen extending from the proximal end to the distal tip, and opening at a distal orifice through the distal tip. The microvalve includes a braided multistrand construct coupled to the distal end of the catheter proximal of the orifice.

The braided multistrand construct is initially formed as a tube. In accord with one embodiment of the infusion device, a primer is unevenly distributed along the strands as a result of primer application or post-application processing. Uneven application can include applying a different thickness of primer over different areas during initial application of primer or post-processing subsequent to an initial preferably consistent application of primer to remove primer from selected regions of the strands. Post-processing can include partial removal or complete removal of the primer from selected areas of the elastic strands. Then, after the uneven application of primer, a polymer filter coating is applied over the elastic strands and across diamond-shaped interstices formed between the braided strands. Whereas the primer is intended to increase adhesion of the filter coating, the uneven distribution of the primer over the elastic strands modifies, and specifically reduces, such adhesion of the filter coating to the strands at locations where the primer is reduced or eliminated.

The braided multistrand construct is naturally biased to radially expand outward and has a proximal end and a distal end. An inside of a first end of the braided construct is coupled to the catheter at a first location proximally adjacent the distal tip of the catheter, the braid is then everted to bend directed back such that the surface formed as the outside of the braided construct is coupled to the catheter at a second location proximally displaced from the first location. The occluder has a shape that flares outward in a proximal to distal direction, and maximizes to a largest diameter. When the occluder is placed in a vessel, antegrade (downstream) pressure from flow deforms the occluder closed to permit flow around the occluder. Then, when a therapy is infused through the catheter, retrograde pressure (in a distal to proximal direction) against the occluder forces the occluder open into contact with the vessel wall of the patient and blocks retrograde flow within the vessel. With reduced adhesion, the elastic strands in the braid are less restrained to each other by the filter coating. As a result, a reduced force is required to move the strands relative to each other, and thus the strands are able more easily and rapidly reconfigure to allow the occluder to move between the open and closed configurations.

In accord with another embodiment of the infusion device, the primer may be evenly or unevenly distributed between the elastic strands and filter coating. Then, the filter coating is selectively removed down to the elastic strands while the braid is in tubular form. The filter coating is preferably removed by laser ablation. In an embodiment, removal is preferably limited to areas distal of where the braid will subsequently reshaped to have a maximum diameter of the occluder. Other embodiments may ablate the filter coating from the tubular braid such that the remaining coating will be located on distal portions of the occluder or be removed from the maximum diameter of the occluder. After the coating is ablated from the selected portions, then the tubular braid is reshaped to form the occluder and attached to the catheter. As a result of the selective removal in the tubular form, precise transitions are generated between the coating and uncoated portions of the braid. This allows the infusion device to operate with consistent filter valve occluder performance when subject to force.

The aspects of uneven distribution of the primer and the ablation of the filter coating from braid in tubular form can be used separately or together. However, each aspect operates to improve the filter valve occluder performance.

### BRIEF DESCRIPTION OF DRAWINGS

Prior art FIG. 1 is a side elevation of a prior art microvalve infusion system.

FIG. 2 is side elevation of a microvalve infusion system according to an embodiment described herein.

FIG. 3 is a schematic illustration of a tubular braided construct for use in the manufacture of the occluder for the microvalve infusion system shown in FIG. 2.

FIGS. 4, 5 and 6 are schematic illustrations of reconfiguration of a pixel of a tubular braid according to the microvalve occluder described herein.

FIGS. 7 and 8 are schematic illustrations of reconfiguration of a tubular braid according to the microvalve occluder described herein.

FIG. 9 is a schematic illustration of the tubular braided construct of FIG. 3 coated in a primer in accord with the teachings herein.

FIG. 10 is a schematic illustration of the tubular braided construct of FIG. 9 coated in a polymer filter coating in accord with the teachings herein.

FIGS. 11 and 12 illustrate manufacture of a microvalve occluder on a catheter from a polymer coated braid dip coated inside a tubular mandrel.

FIG. 13 is a partial section view of the polymer coated braided construct mounted on a catheter in the manufacture of the occluder for the microvalve infusion system.

FIG. 14 is a view similar to FIG. 13, also showing the polymer coating removed from a distal portion of the occluder after mounting on the catheter.

FIG. 15 is a view similar to FIG. 10, showing a portion of the polymer coating removed from the tubular braided construct by laser ablation.

FIG. 16 is a partial section view of the polymer coated braided construct of FIG. 15 mounted on a catheter in the manufacture of the occluder for the microvalve infusion system.

### DETAILED DESCRIPTION OF THE PREFERRED EMBODIMENTS

With reference to the human body and components of the devices and systems described herein which are intended to be hand-operated by a user, the terms “proximal” and “distal” are defined in reference to the user's hand, with the term “proximal” being closer to the user's hand, and the term “distal” being further from the user's hand, unless alternate definitions are specifically provided.

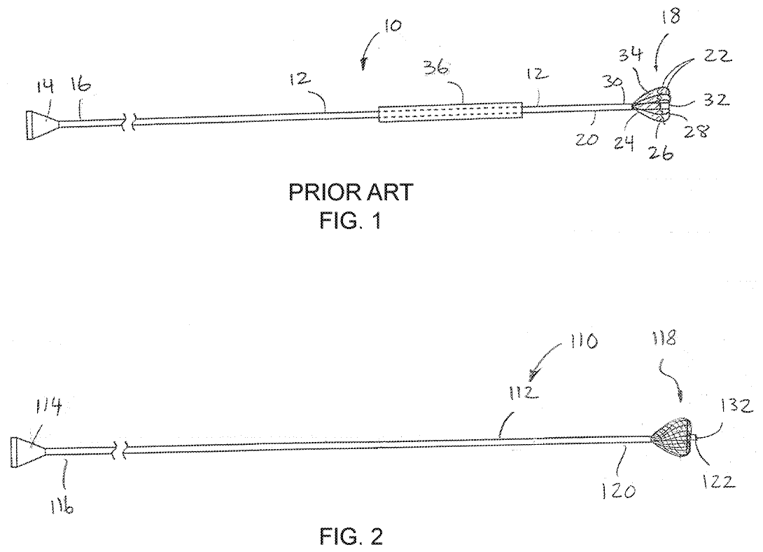

Turning now to FIG. 2, an infusion device 110 is provided that includes a catheter 112 and a microvalve occluder 118. The catheter 112 has a proximal end 116, a distal end 142 with a distal tip 122, and an infusion lumen extending from the proximal end to the distal tip, and opening at a distal orifice 132 through the distal tip. The microvalve occluder 118, which is described in detail below, is formed from a polymer-coated, braided, multistrand tubular construct that is coupled in a preferred manner to the distal end 142 of the catheter, proximal of the orifice 132.

The catheter 112 is between two and eight feet long, and has an outer diameter of between 0.67 mm and 3 mm (corresponding to catheter sizes 2 French to 12 French), and is made from a liner made of fluorinated polymer such as polytetrafluoroethylene (PTFE) or fluorinated ethylene propylene (FEP), a braid made of metal such as stainless steel or nickel titanium alloy, or a polymer such as polyethylene terephthalate (PET) or liquid crystal polymer, and an outer coating made of a polyether block amide thermoplastic elastomeric resin such as Pebax®, polyurethane, polyamide, copolymers of polyamide, polyester, copolymers of polyester, fluorinated polymers, such as PTFE, FEP, polyimides, polycarbonate or any other suitable material, or any other standard or specialty material used in making catheters used in the bloodstream.

A hub 114 is preferably coupled to the proximal end 116 of the catheter 112. The hub 114 may include a leur connector or other standardized connector. The infusion lumen extends into the hub 114 so that the hub is adapted for delivery of a therapeutic agent from outside the body of the patient (not shown) to a target vessel (artery or vein) in the patient. The hub 108 is preferably also adapted to facilitate advancement of a guidewire through the infusion lumen and/or coupling of a syringe for infusion of a therapeutic through the infusion lumen. Any hub suitable for at least facilitating delivery of a therapeutic into the infusion lumen can be utilized.

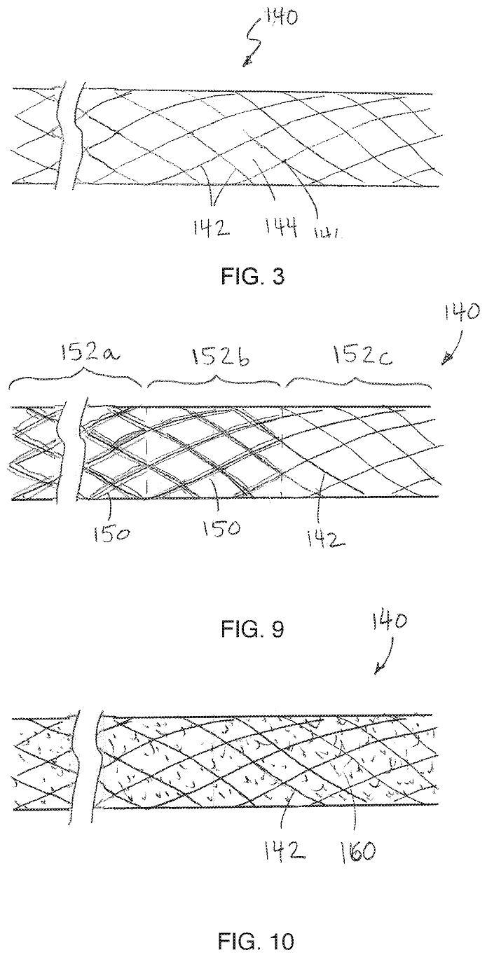

The microvalve occluder 118 is preferably made as now described. Turning to FIG. 3, a braid of multiple strands in tubular form 140 is created or provided. The tubular braid 140 is comprised of multiple metal (e.g., stainless steel or nickel-titanium alloy) or polymer filaments or strands 142 in a tubular braided construction, which form a substantially closed shape when deployed and not subject to outside forces. Where polymeric filaments are utilized, the filaments 142 may be composed of PET, polyethylene-napthalate (PEN), liquid crystal polymer, fluorinated polymers, nylon, polyamide or any other suitable polymer. If desired, when polymeric filaments are utilized, one or more metal filaments may be utilized in conjunction with the polymeric filaments. According to one aspect of the invention, where a metal filament is utilized, it may be of radio-opaque material to facilitate tracking the filter valve occluder 110 and its configuration within the body. The filaments 142 are not bonded to each between their ends so to enable them to move relative to each other between their ends. The filaments are spring biased (i.e., they have “shape memory”) to assume a desired crossing angle relative to each other. Between each two adjacent sets of crossing filaments, diamond-shaped interstices or pixels 144 are formed, with the crossing of the filaments defining vertices 146 of the pixels. The specific shape and size of the pixels is determined by the braid angle between crossing filaments in the tubular braid.

The diameter of the filaments 142 are chosen in the range of 0.025 mm to 0.127 mm, although other diameters may be utilized. Preferably, the pitch angle (i.e., the crossing angle assumed by the braided filaments in the fully open deployed position) is chosen in the range of 100° to 150°, although other pitch angles may be used.

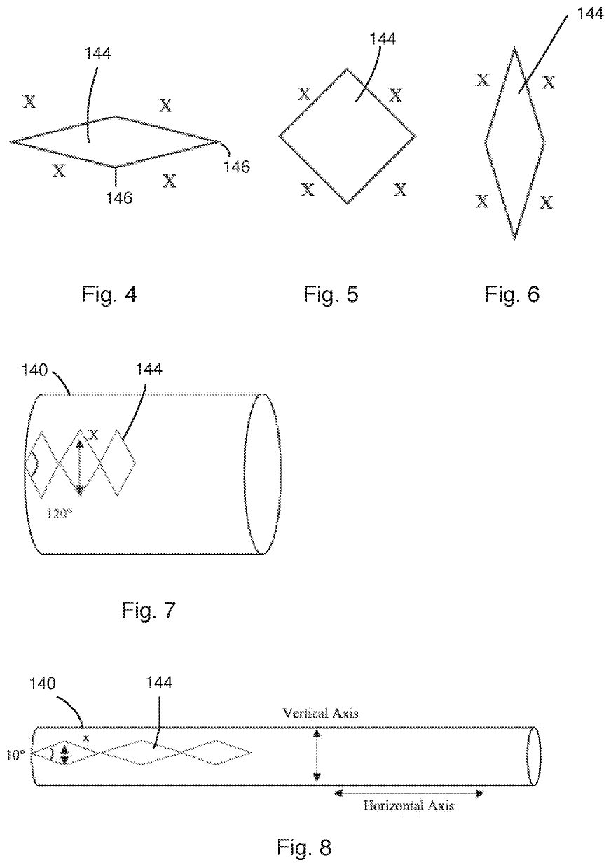

More particularly, the tubular braid 140 operates to expand and contract radially as it is displaced longitudinally. The radial force the braid exerts is related to the bending strength of the filaments 142 making up the braided structure and the crossing angle at which the braid filaments intersect at the vertices 146. The closer a filament 142 is to a vertical orientation within the braided structure, the more radial force it will exert. As the tubular braid 140 is displaced laterally (expanded to compressed), the vertices 146 of the pixels of the pixels 144 can move, but the length of the sides (X) of each pixel 144 is fixed. See FIG. 4 (pixel 144 of expanded tubular braid), FIG. 5 (pixel of neutral tubular braid), and FIG. 6 (pixel of compressed tubular braid).

Referring to FIG. 7, in one example, a tubular braid 140 is set with pixels 144 having a braid angle of 120°. The vertical axis of the pixel 144 will have a length of 1.732*X calculated by 2*sin(120/2)*X, and the horizontal axis of the pixel 144 will have a length of 1*X calculated by 2*sin((180−120)/2)*X. Then, when the tubular braid 140 is elongated lengthwise to reduce the diameter of the structure, like when the braid is introduced through a narrow catheter, the pixel is elongated along the horizontal axis and compressed in the direction of the vertical axis, as shown in FIG. 8. The vertical length is reduced to 0.1743*X as calculated by 2*sin(10/2)*X, and the horizontal length is elongated to 1.992*X as calculated by 2*sin((180−10)/2)*X. This is approximately a 10-fold compression in vertical length as calculated by 1.732/0.1743=9.93 and an approximately two-fold elongation increase in the horizontal axis as calculated by 1.992/1=1.992. Any material and material coating process used over the braid and within the pixel must allow for this simultaneous degree of elongation or compression without failure.

The radial forces of expansion of the tubular braid is described by Jedwab and Clerc (Journal of Applied Biomaterials, Vol. 4, 77-85, 1993) and later updated by DeBeule (DeBeule et al., Computer Methods in Biomechanics and Biomedical Engineering, 2005) as:

$$F = 2 n [ \frac{{GI}_{p}}{{K}_{3}} ( \frac{2 sin β}{{K}_{3}} - {K}_{1} ) - \frac{EI tan β}{{K}_{3}} ( \frac{2 cos β}{{K}_{3}} - {K}_{2} ) ]$$

where K₁, K₂, K₃ are constants given by:

$${K}_{1} = \frac{sin 2 {β}_{0}}{{D}_{0}} {K}_{2} = \frac{2 {cos}^{2} {β}_{0}}{{D}_{0}} {K}_{3} = \frac{{D}_{0}}{cos {β}_{0}}$$

and I and Iₚ are the surface and polar moments of inertia of the braid filaments, E is the Young's modulus of elasticity of the filament, and G is the shear modulus of the filament. These material properties along with the initial braid angle (β₀), final braid angle (β), tubular braid diameter (D₀), and number of filaments or strands (n) impact the radial force of the braided valve.

As will be appreciated by those skilled in the art, the braid geometry and material properties of the filaments 142 are intimately related to the radial force and time constant of the filter valve. Since the filter valve is useful in a variety of vessels of different diameters and flow conditions, each implementation can have a unique optimization.

In one exemplar embodiment, the braid uses twenty-four (24) strands 142: twelve (12) nickel titanium strands 142 having a diameter of approximately 0.02 mm (0.0008 inch) and twelve (12) nickel titanium strands 142 having a diameter of approximately 0.032 mm (0.00125 inch). This produces a tubular braid 140 having approximately 34 to 38 pixels per linear inch and a braid angle between 120° and 130° when the diameter of the tubular braid 140 is set at 4.5 mm.

In another exemplar embodiment, the braid uses twenty-four (24) strands 142: twelve (12) nickel titanium strands 142 having a diameter of approximately 0.025 mm (0.001 inch) and twelve (12) nickel titanium strands 142 having a diameter of approximately 0.038 mm (0.0015 inch). This produces a tubular braid 140 having approximately 27 to 31 pixels per linear inch and a braid angle between 120° and 130° when the diameter of the tubular braid 140 is set at 6 mm.

In accord with one aspect of the infusion device and method, referring to FIG. 9, a primer 150 is unevenly distributed to the strands 142 of the tubular braid 140. Uneven distribution of the primer can include applying a different thickness of primer over different areas of the tubular braid during initial application or post-processing subsequent to an initial preferably consistent application to remove primer from selected regions of the strands while the braid is in tubular form. The different applied thickness can include reducing the thickness of the primer along one or more portions of the braid. The different applied thickness can include completely removing the primer along one or more portions of the braid.

The primer may be applied by spray coating, dip coating, brushing, or any other suitable application method. For uneven initial application, a heavier covering or multiple coats of primer may be applied in selected areas, and a lesser coating or even no coating may be applied at other selected areas. FIG. 9 illustrates area 152a in which the strands 142 are provided with a thicker application of primer 150, area 152b in which the strands are provided with a thinner application of primer, and area 152c in which the strands are provided with no primer. The tubular braid 140 does not necessarily have all such areas, but in accord with the present embodiment will have area 152a and at least one of areas 152b and 152c.

Post-processing can include partial removal or complete removal of the primer from selected areas of the elastic strands. For uneven distribution via post-processing, the primer may be mechanically fully or partially removed in thickness from over selected areas. Mechanical removal can include scraping. The primer may alternatively or additionally be removed via non-mechanical means such as via laser ablation or heating and pealing. Any other suitable means for reducing, partially removing, or fully removing the primer from selected areas of the tubular braid after the primer has already been applied can be utilized.

Turning now to FIG. 10, after the primer 150 has been modified to reduce the thickness of the primer in the selected areas, a polymer filter coating 160 is applied over the primed and/or remaining uncoated elastic strands 142 and across the pixels 144 formed between the strands. The braid is preferably set in a neutral position in order to receive the coating 160; that is, it is neither longitudinally compressed nor elongated. The polymer 160 can be coated onto the braid by any of several methods, including by spraying, spinning, electrospinning, bonding with an adhesive, thermally fusing, mechanically capturing the braid, melt bonding, dip-coating, or any other desired method, to form a coating suitable for use as a filter. The filter can either be a material with pores such as ePTFE, a solid material that has pores added such as polyurethane with laser drilled holes, or the filter can be a web of very thin filaments that are laid onto the braid.

Where the polymer filter 160 is a web of thin filaments, the characteristic pore size of the filter can be determined by attempting to pass beads of different diameters through the filter and finding which diameter beads are capable of passing through the filter in large quantities. The very thin filaments can be spun onto a rotating mandrel according to U.S. Pat. No. 4,738,740 with the aid of an electrostatic field or in the absence of an electrostatic field or both. The filter thus formed can be adhered to the braid structure with an adhesive or the braid can be placed on the mandrel and the filter spun over it, or under it, or both over and under the braid to essentially capture it. The filter can have some pores formed from spraying or electrospinning and then a secondary step where pores are laser drilled or formed by a secondary operation. In one embodiment a material capable of being electrostatically deposited or spun is used to form a filter on the braid, with the preferred material being capable of bonding to itself. The filter may be made of polyurethane, thermoplastic urethanes such Pellethane®, polyolefin, polyester, fluoropolymers, acrylic polymers, acrylates, polycarbonates, silicone, or other suitable material. The polymer is spun onto the braid in a wet state, and therefore it is desirable that the polymer be soluble in a solvent. In the preferred embodiment, the filter is formed from polyurethane in a dimethylacetamide (DMA) and tetrahydrofuran (THF) solution. The polymer in solution is spun, with a preferred concentration of 5-10% solids for an electrostatic spin process and 15-25% solids for a wet spin process.

As another alternative construct for polymer-coating 160 the braid, the braid can be dip-coated to form a filter onto the braid. The braid is mounted on a mandrel having the same outer diameter as the inner diameter of the fully expanded braid. The mandrel can be polytetrafluoroethylene (PTFE)-coated steel, in which the PTFE acts as a release surface. Alternatively, a non-coated mandrel may be used. It is important that inner diameter of the braid and the outer diameter of the mandrel not be spaced from each other when the braid is mounted on the mandrel. Thus, they preferably have a common diameter within a tolerance of ±0.065 mm. Keeping the entire inner braid in contact with the mandrel allows for the filaments to be evenly coated with the polymer, as subsequently described, so that the filter valve expands uniformly after the polymer dries. Alternately, the tubular braid 140 can be mounted on an oversized mandrel (greater than the inner diameter of the braid), but such will result in an increase in the braid angle of the filaments, and thereby resize the filter valve and effect the expansion force thereof. In an alternate arrangement the braid may be mounted within a tubular mandrel having the same size as the outer diameter of the braid, provided with like tolerances described above. As yet another alternative, the braid can be mounted inside an undersized tubular mandrel (having an inner diameter smaller than the outer diameter of the braid), but such will result in a decrease in the braid angle of the filaments, and thereby also resize the filter valve and effect the expansion force thereof.

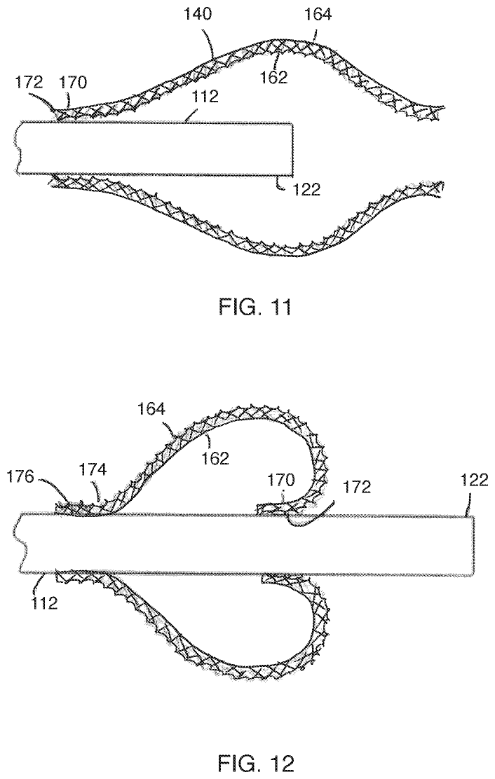

The type of mandrel (solid or tubular), and placement of the braid relative to the mandrel affects localization of the polymer on the braid. For example, when the braid is mounted on the exterior of a mandrel and dip coated, the braid has a resulting polymer coating that has a smooth interior surface (which previously faced the mandrel) and a rougher exterior surface. To the contrary, when the braid is mounted on the interior surface of a tubular mandrel and dip coated, the braid has a resulting polymer coating that has a smooth exterior surface (which previously faced the mandrel) and a rougher interior surface. In general, the smoother surfaces provided enhanced sealing capability against a vessel surface, whereas the rougher surfaces, defined by peaks (high points of the filamentary structure) and valleys (low points between the filaments with gaps in the polymer), presents lower friction against the vessel wall. In particular, the rougher, lower friction surface has enhanced lubricity and is better adapted for an occluder requiring contact with and translational relative to the vessel wall. See the coated tubular braid shaped into an occluder, described below with respect to FIGS. 11 and 12, for further illustration.

Once the braid 140 is tightly mounted on (or within) the mandrel, the braid is dip coated into a polymer solution at a controlled steady rate. The solution is an elastomeric thermoplastic polymer dissolved in a solvent system with a boiling point ranging from 30-200° C. to produce a solution with a dynamic viscosity range of 50-10,000 cP. The rate of decent and accent is inversely dependent upon the viscosity of the solution and ranges from 1-100 mm/sec. The rate is critical to provide an even coating of the polymer on the braid, to allow wetting of all surfaces of the braid even at locations where the braid filaments are in contact with the mandrel and consequent wicking of the polymer coating into the braid particularly to the surface in contact with the mandrel, and to release air bubbles that may be trapped during the dipping process. By way of example, in one embodiment of the method for dipping into a thermoplastic urethane solution (for example, Pellethane® dissolved in the solvents dimethylacetamide (DMA) and tetrahydrofuran (THF)), the rate is such that the dwell time of a 135 mm (6 inch) braid is 16 seconds. The rate is also preferably such that the polymer wicks down the length of the entire braid during withdrawal of the braid from the solution. The braid is dipped one time only into the solution to limit the thickness of the coating and thereby prevent restraint on the braid filaments and/or control smoothness of the polymer coating membrane. The controlled rate may be controlled by coupling the mandrel to a mechanized apparatus that dips and raises the braid on the mandrel at the steady and controlled rate into the polymer solution.

After the braid 140 is withdrawn from the polymer solution, the solvent is evaporated over a time frame relative and temperature range corresponding to the solvent boiling point, with higher temperatures and longer durations utilized for high boiling point solvents. All preferred polymer solutions use some DMA to control the uniformity of the coating thickness, and may use THF to control the rate of solvent evaporation. The ratio of high boiling point solvents such as DMA to low boiling point solvents such as THF allows for control over the rate of transition from a lower viscosity high solvent content polymer solution to a high viscosity low solvent content polymer solution to a solid solvent free material, affecting the quality of the polymer membrane. In one method, the solvents are released in an oven heated to a temperature above the boiling point of DMA (165° C.) in order to rapidly release the DMA. A preferred time of heating at this temperature is 5 minutes which is sufficient to release the DMA. It is appreciated that THF has a substantially lower boiling point (66° C.) and will vaporize quickly without such substantial heating. Alternatively, the polymer-coated braid can be oven heated at a temperature below the boiling point of DMA, e.g., 80° C.-100° C., which will release of the DMA from the coated braid, but at a slower rate than would occur above the boiling point of DMA. This temperature rapidly drives off the DMA while maintaining the integrity of the coated braid. A preferred time of heating at this temperature is 10 minutes which is sufficient to release the DMA. As yet another alternative, the polymer-coated braid can be allowed to dry ambient room temperature, which results in DMA release occurring at a slower rate than each of the above.

After the solvents have been released from the polymer-coated braid, the coated braid is cooled. Once cooled, the coated braid is released from the mandrel. If the mandrel is coated with PTFE, the braid may self-release from the mandrel or may be readily released. If the mandrel is uncoated, a release agent such as isopropyl alcohol (IPA) may be used to facilitate removal of the coated braid from the mandrel. The resulting elastomeric membrane filter formed on the braid may be elastically deformed over a range of 100-1000% elongation. In addition to Pellethane®, the membrane may be formed from, but is not limited to, other polyether-based aromatic thermoplastic urethanes, polyether-based aliphatic thermoplastic urethanes (e.g., Tecoflex®), polyether block amides (e.g., Pebax®), styrene-isoprene-butadiene-styrene (SIBS), silicone, and other polymers. These polymers may be dissolved in appropriate solvents or heated to their melting point to form a fluid.

The material for the coating 160 should be elastic, such that it is able to fully recover from a two to three fold elongation and ten-fold compression. The coating thickness should be minimized so that the material volume does not significantly impact the braid structure during compression; if the coating is too thick it will excessively bunch as it is compressed. The strain required to elongate the coating should be sufficiently low so that it does not substantially bend the filaments of the braid. If the elongation force is greater than the bending strength of the braid, the structure will bend and fold as opposed to changing in radial diameter during elongation.

In an exemplar embodiment, the coating 160 is an 80A Shore hardness thermoplastic elastomer, which has a tensile modulus of 6.10 MPa at 100 percent elongation, a tensile modulus of 10.3 Mpa at 300 percent elongation, can elongate at least 500 percent before breakage, and is applied to produce a coating thickness of between 5 μm to 20 μm. The exemplar coating can be a polyether-based aromatic thermoplastic urethane such as Pellethane®. These coating parameters can be used, without limitation, in combination with each of the exemplar braid construction embodiments described above.

It is further preferred that the coating 160 be adapted to withstand a Kv/h ratio of greater than three, where Kv/h is the ratio of vertical compression to horizontal elongation, and where the vertical compression (v) is at least 10 and the horizontal elongation (h) is at least 3.

If is also preferred that the coating 160 be adapted to have a Kb/m ratio of 1 to 6, where Kb/m is the ratio of diameter of the strand (b) to the thickness of the coating (m), and where a preferred strand thickness is 20-40 μm and a preferred coating thickness is 5-20 μm.

Depending on the polymer and coating technique, the coating can be fluid impermeable or porous. If porous, the coating can have a characteristic pore size between 10 μm and 500 μm, or more preferably between 15 μm and 100 μm, or even more preferably, less than 40 μm and yet more preferably between 20 μm and 40 μm.

In accord with various embodiments, the polymer coating 160 is located over strands 142 that have higher primer thickness (area 152a), lower primer thickness (area 152b), and/or no primer (area 152c). In accord with embodiments, the polymer coating is unevenly applied between the proximal and distal ends of the braided tubular form.

The polymer-coated braid is also preferably provided with a hydrophilic coating, hydrophobic coating, or other coating that affects how proteins within blood adhere to the filter. More specifically, the coating is resistant to adhesion of blood proteins. Suitable coatings include ANTI-FOG COATING 7-TS-13 from Hydromer, Inc. of Branchburg, NJ, and SERENE COATING from Surmodics, Inc, of Eden Prairie, MN. These and other coatings can be applied to the filter by, e.g., dipping, spraying, or roll or flow coating.

In view of the uneven distribution of the primer over the elastic filamentary strands, the adhesion from the filter coating to the strands is modified relative to known coatings on braids. Specifically, whereas the primer is intended to increase adhesion of the filter coating, areas with reduced or no primer permit the braid to move more easily under the coating; reduced force is required to deform the filter coating at such locations. Consequently, a lower change in intravessel pressure is required to change the shape of the braided structure at such location to allow intended passage of fluid or rapidly prevent unintended passage of fluid and infusates.

The areas of reduced primer dynamically operate more in accord with a tubular braid without any coating. On the other hand, the areas of the braid with the primer coating will be affected by the characteristics of the coating; i.e., the location of the coating, the stiffness of the specific coating, and the thickness of the applied/remaining coating. For example, a primer made of a stiff material will stiffen the length of individual filaments and bind the intersections of the crossing filaments on the braid. The force then required to bend a filament is then associated with the bending force of the filament over a length equivalent to the length over a side of a pixel. Stiffer materials include potential primer coatings like polyamide and parlyene, whereas more elastic primer coatings such as polyether-based aromatic and aliphatic thermoplastic urethanes will have a reduced effect on the bending force. In addition, the thinner the coating for a given primer, the less impact the primer will have on the braid properties.

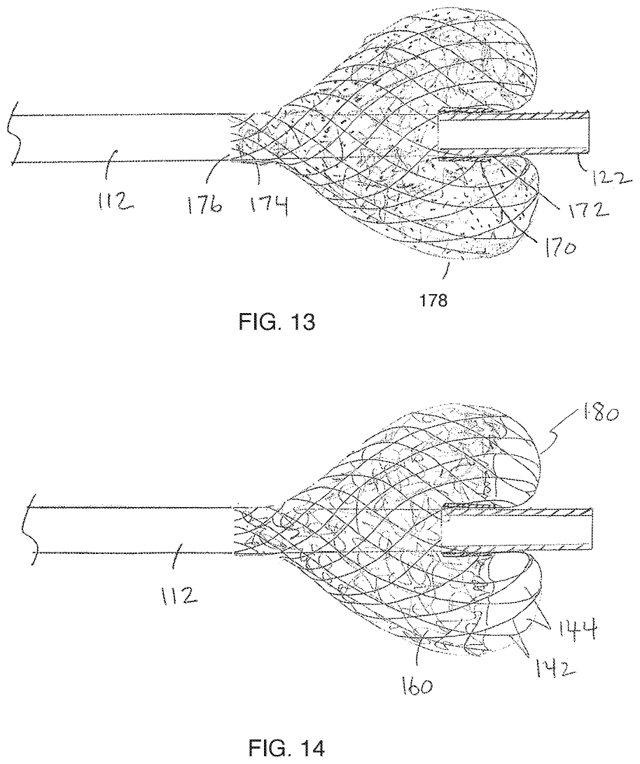

Turning now to FIG. 11, after the tubular braid 140 is polymer coated, it is ready for assembly to the catheter 112. More particularly, the plurality of braided strands 142, the primer 150 and the polymer filter 160 in tubular form 140 having an rough surface 162 initially facing inward and a smoother surface 164 initially facing outwards. A first end 170 of the tubular form 140 is fixed to the catheter 112 at a first location 172 proximally adjacent the distal tip 122 of the catheter, with the rougher surface 162 facing inward attached to the catheter, and the smooth surface 164 facing outward. Then, referring to FIGS. 12 and 13, the tubular form 140 is reshaped by everting the tubular form and bending it back such that the smooth surface 164 now faces inward, and rough surface 162 now faces outward. The second end 174 of the tubular form 140 is then coupled to the catheter at a second location 176 proximally displaced from the first location 172 to define a shape that flares outward in a proximal to distal direction and maximizes to a largest diameter at a central portion 178. In the flared form, the braided strands or filaments define pixels of different sizes. The pixels 144 at the proximal end of the filter valve are elongated and have lower radial force, whereas the pixels in the larger diameter portions are longitudinally compressed and having a larger radial force; i.e., an increased radial force where the microvalve occluder is intended to seal relative to the vessel wall.

Turning to FIG. 14, once the tubular form is attached to the catheter 112 in the occluder shape, the polymer coating 160 is preferably removed from a distal portion 180 of the occluder to open the occluder between the strands 142 thereat; i.e., such that fluid can flow through and into the occluder at the open pixels (interstices) 144 in at least a portion of the distal portion. The removal of the polymer 160 from occluder is preferably by solvents, laser ablation or mechanical means. However, due to the shape of the occluder on the catheter and the catheter extending through the occluder, there may be some roughness at the perimeter of the removed polymer coating.

In accord with embodiments, the primer 150 under the polymer coating 160 is thicker at the proximal end of the occluder and thinner or non-existent under the distal end of the occluder. In accord with embodiments, the primer 150 under the polymer coating 160 is thicker proximal of a maximum diameter of the occluder 118 and thinner distal of the maximum diameter of the occluder.

When the occluder 118 is placed in a vessel, antegrade (downstream) pressure from flow deforms the occluder closed to permit flow around the occluder. Then, when a therapy is infused through the infusion lumen and out the distal orifice 132 of the catheter 112, retrograde pressure (in a distal to proximal direction) against the occluder 118 forces the occluder 118 open into contact with the vessel wall of the patient and blocks retrograde flow within the vessel. With reduced adhesion from the modified primer application, the elastic strands 142 in the braid are less restrained to each other by the filter coating 160. As a result, a reduced force is required to move the strands 142 relative to each other, and thus the strands are able more easily and rapidly reconfigure to allow the occluder to move between the open and closed configurations.

More particularly, when subject to an infusion pressure at the distal orifice 132 of the catheter 112, the filter valve occluder 118 moves between deployed positions allowing downstream fluid passage (closed) and preventing fluid passage (open) in a static fluid (e.g., glycerin) having a viscosity approximately equal to the viscosity of blood (i.e., approximately 3.2 cP) in 0.067 second. For purposes herein, the time it takes to move from the closed position to the open position in a static fluid is called the “time constant”. According to another aspect of the invention, the filter valve 118 is arranged such that the time constant of the filter valve occluder 118 in a fluid having the viscosity of blood is between 0.01 seconds and 1.00 seconds. More preferably, the filter valve occluder 118 is arranged such that the time constant of the filter valve in a fluid having the viscosity of blood is between 0.05 and 0.50 seconds. The time constant of the filter valve occluder 118 may be adjusted by changing one or more of the parameters described above (e.g., the number of filaments, the modulus of elasticity of the filaments, the diameter of the filaments, etc.).

The deployed filter valve 118 opens and closes sufficiently quickly to achieve high capture efficiency of therapeutic agents in the presence of rapidly changing pressure conditions. More particularly, when pressure at the distal orifice 132 increases higher than the pressure in the blood vessel, the seal between the periphery of the filter valve and the vessel wall is increased, thus blocking refluxing therapeutics. It is important to note that pressure is communicated throughout the vasculature at the speed of sound in blood (1540 m/s) and that the valve opens and closes in in response to pressure changes within the blood vessel. Since the expandable filter valve responds to pressure changes, it reacts far faster than the flow rates of therapeutics in the blood (0.1 m/s) thereby preventing reflux of any therapeutics.

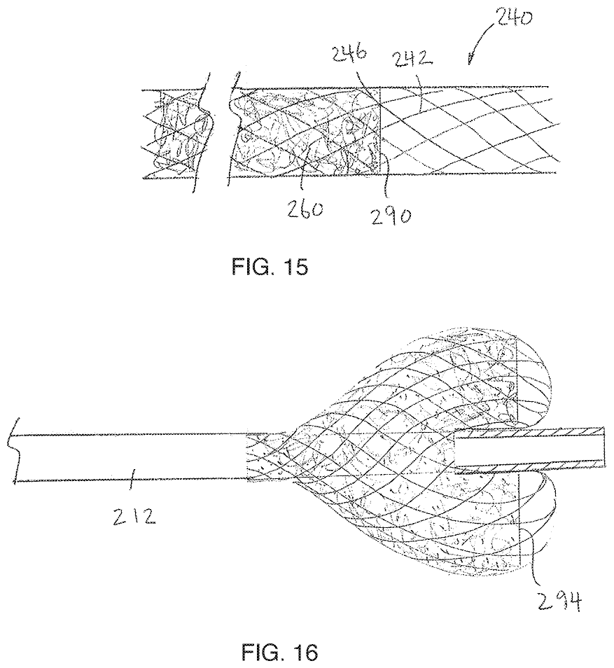

Turning now to FIGS. 15 and 16, according to another embodiment, the primer 250 may be applied to the braided strands 242 in either a normal even coating over the elastic strands, or in the uneven coating described above. The filter coating 260 is then applied according to any suitable method described herein. Then, after application of the filter coating on the strands, a portion of the filter coating 260 is selectively removed down to the elastic strands 242 while the braid is still in tubular form 240. In an embodiment, the filter coating is removed from areas of the tubular braid distal of where the braid will subsequently assume its maximum diameter when reshaped as the occluder. The tubular form 240 defines a circular circumference 290, and in an embodiment the polymer coating 260 is removed along and to one side of the circumference. In another embodiment, the polymer coating may be removed in an area defined between two circumferences displaced along the tubular form. In another embodiment, the polymer coating is removed to one side of a ring of vertices 246 extending about a circumference of the tubular form, with the demarcation between polymer coating and coating removal extending through the vertices. In another embodiment, the polymer coating is removed along a curving line extending a perimeter of the tubular form. The filter coating is preferably removed by laser ablation as such provides a sharp clean demarcation between polymer-coated and uncoated areas on the braid.

Then, after the coating 260 is ablated from only the selected portions on the braided tubular form 240, the tubular braid is reshaped to form the occluder 218 and attached to the catheter 212 as described above. As a result of the selective removal of the polymer coating 260 while the braided strands 242 are in the tubular form, a precise transition 294 is generated between the polymer coated and uncoated portions of the final occluder 218. Such precision is possible as the coating is ablated while the braid is in a regular tubular form, and off the catheter for better handling. This allows the resulting infusion device to operate with consistent filter valve occluder performance.

In addition, removal of the polymer filter coating via laser ablation while in tubular form can be effective for occluders or even other devices having final shapes other than as shown. Further, depending on the application, the polymer filter coating may be removed at the proximal end of the device rather than at the distal end.

There have been described and illustrated herein embodiments of devices and methods for manufacturing devices for use in delivering therapeutic agents within a vessel. While particular embodiments of the invention have been described, it is not intended that the invention be limited thereto, as it is intended that the invention be as broad in scope as the art will allow and that the specification be read likewise. Thus, while various materials have been listed for the microvalve filaments, the polymer coatings, and the catheter, it will be appreciated that other materials can be utilized for each of them in each of the various embodiments in combination and without limitation. While partial primer removal and partial polymer coating removal have been described with respect to an exemplar occluder having a specific in which a portion of the occluder is inverted/everted, these concepts are applicable to multistrand occluders of any shape and wherein the coating can be removed from removed from any portion of the occluder. In addition, while the concepts have been described with respect to braided occluders, the concepts of partially removing primer and polymer coating removal prior to shaping the occluder are also applicable to multistrand elastic occluders having a partially non-braided construction, such as at the distal portion, or fully non-braided constructs. Also, various methods for partial primer removal and partial coating removal have been described, such methods are not intended to be an exclusive listing, but rather exemplary. Also, while the invention has been described with respect to use in association with a vascular therapeutic device for delivery of a therapeutic to a vessel of a patient, the manner of controlling application of the primer and modifying the polymer coating prior to forming the occluder can be useful in other medical treatment and implantable devices and for other therapeutic applications, and such are considered within the scope hereof. In addition, while several examples of braided tubular constructs for use in the manufacture of the microvalve occluder have been disclosed, it will be appreciated that other constructions with variations in one or more of the materials, dimensions, and number of filaments, braid angle, and overall dimension of the construct can be utilized. Where the term “approximately” or “substantially” is used to modify a number herein, such term means within 10 percent of the modified number. It will therefore be appreciated by those skilled in the art that yet other modifications could be made to the provided invention without deviating from its scope as claimed.

What is claimed is:

1. A therapeutic device for delivery of a therapeutic agent within a vessel of a patient during a vascular procedure, comprising:
a) a catheter having a proximal end and a distal end with a distal tip, and an infusion lumen extending from the proximal end to the distal end; and
b) an occluder coupled to the distal end of the catheter, the occluder having a first end and a second end, the occluder comprising,
i) a plurality of strands extending in a tubular arrangement from the first end to the second,
ii) a primer of a first polymer unevenly distributed to the plurality of elastic strands, and
iii) a second polymer different than the first polymer provided as a coating over at least a portion of the elastic strands and extending between the strands.

2. The therapeutic device according to claim 1, wherein:
the plurality of elastic strands extending in the tubular arrangement includes braided strands which cross and define diamond-shaped interstices in spaces between the braided strands.

3. The therapeutic device according to claim 2, wherein:
the primer is distributed in different thicknesses over different portions of the braided strands.

4. The therapeutic device according to claim 2, wherein:
the primer is completely absent from at least a first portion of the braided strands, and the first second polymer is provided as a coating over the first portion.

5. The therapeutic device according to claim 1, wherein:
the occluder has a proximal portion, a distal portion, and a maximum diameter between the proximal and distal portions, and the polymer primer is unevenly distributed to the plurality of elastic strands between the proximal and distal portions.

6. The therapeutic device according to claim 1, wherein:
the occluder has a proximal portion, a distal portion, and a portion with a maximum diameter between the proximal and distal portions, and the primer is unevenly distributed to the plurality of elastic strands between the proximal portion and the portion with the maximum diameter.

7. The therapeutic device according to claim 2, wherein:
the braided strands, primer and coating are in a tubular form having an inner surface and an outer surface, the tubular form fixed to the catheter at a first location proximally adjacent the distal tip of the catheter, everted back such that the inner surface forms an exterior of the occluder, and is coupled to the catheter at a second location proximally displaced from the first location to define a shape that flares outward in a proximal to distal direction.

8. The therapeutic device according to claim 7, wherein:
the coating is removed from a distal portion of the tubular form.

9. A method of manufacturing an occluder for therapeutic device for temporary use within a vessel of a patient during a vascular procedure, the method comprising:
a) obtaining a tubular arrangement of a plurality of elastic strands, the tubular arrangement having first and second ends and defining diamond-shaped interstices in spaces between the braided strands;
b) applying a primer to the tubular arrangement of elastic strands to obtain a primered tubular arrangement of elastic strands, wherein the primer is unevenly distributed between the first and second ends;
c) coating the primered tubular arrangement in a polymer coating to obtain a polymer-coated tubular arrangement of the plurality of elastic strands in which the polymer coating extends across the interstices; and
d) then coupling the polymer-coated tubular arrangement of the plurality of elastic strands to a catheter, the catheter having a proximal end, a distal end, a lumen extending from the proximal end to the distal end, and an outer surface, the tubular arrangement of the plurality of elastic strands attached to the outer surface at the distal end such that the tubular arrangement of the plurality of elastic strands forms the occluder, the occluder adapted to expand into contact with the vessel of the patient during use.

10. The method according to claim 9, further comprising:
after coating and before coupling, removing a portion of the polymer coating from the coated multistrand braid.

11. The method according to claim 10, wherein the tubular form has a circumference, and the removing occurs along and to one side of the circumference.

12. The method according to claim 10, wherein the removing is performed by laser ablation.

13. The method according to claim 10, wherein the occluder has a maximum diameter, and all of the removing occurs distal of the maximum diameter.

14. The method according to claim 10, wherein the catheter includes a hub at its proximal end for attaching a source of a therapeutic agent in fluid communication with the infusion lumen.

15. A method of manufacturing an occluder for a therapeutic device for temporary use within a vessel of a patient during a vascular procedure, the method comprising:
a) obtaining a multistrand braid in tubular form, the tubular form having a first end and a second end;
b) coating the multistrand braid in a polymer coating to obtain a coated multistrand braid;
c) while the coated multistrand braid is in tubular form, removing a portion of the polymer coating from the coated multistrand braid; and
d) then coupling the coated multistrand braid to an infusion catheter, the infusion catheter having a proximal end, a distal end, an infusion lumen extending from the proximal end to the distal end, and an outer surface, the coated multistrand braid attached and reshaped to the outer surface at the distal end such that the multistrand braid forms the occluder, the occluder adapted to expand into contact with the vessel of the patient during use.

16. The method according to claim 15, wherein the tubular form has a circumference, and the removing occurs along and to one side of the circumference.

17. The method according to claim 16, wherein the removing is performed by laser ablation.

18. The method according to claim 15, wherein the occluder has a maximum diameter, and all of the removing occurs distal of the maximum diameter.

19. The method according to claim 16, wherein the infusion catheter includes a hub coupled at the proximal end for coupling a source of a therapeutic agent in fluid communication with the infusion lumen.

20. A therapeutic device for delivery of a therapeutic agent within a vessel of a patient during an vascular procedure, comprising:
a) a catheter having a proximal end and a distal end with a distal tip, and an infusion lumen extending from the proximal end to the distal end; and
b) an occluder coupled to the distal end of the catheter, the occluder having a first end and a second end, the occluder comprising,
i) twelve nickel-titanium elastic strands having a diameter of approximately 0.025 mm and twelve nickel titanium strands having a diameter of approximately 0.038 mm, the elastic strands formed in a tubular braided having approximately 27 to 31 pixels per linear inch and a braid angle between 120° and 130° when the diameter of the tubular braid is at approximately 6 mm, and
ii) a coating over at least a portion of the elastic strands.

21. The therapeutic device of claim 20, wherein:
the tubular braid has an inner surface and an outer surface and the tubular form fixed to the catheter at a first location proximally adjacent the distal tip of the catheter, everted back such that the inner surface forms an exterior of the occluder, and is coupled to the catheter at a second location proximally displaced from the first location to define a shape that flares outward in a proximal to distal direction, the exterior of the occluder having a rougher surface than the interior of the occluder.

22. The therapeutic device of claim 21, wherein:
the exterior of the occluder has more surface peaks and valleys than the interior of the occluder.

23. The therapeutic device of claim 21, wherein:
the coating is a polymer coating.

24. The therapeutic device of claim 23, wherein:
the coating is between 5-20 μm in thickness.
